(19) United States
(12) Patent Application Publication
(10) Pub. No.: US 2026/0350774 A1
(43) Pub. Date: Nov. 5, 2026

(54) METHOD FOR AUTOMATING PETROLOGICAL INTERPRETATION USING GENETIC INTERPRETATION LOGIC

(71) Applicant: Petróleo Brasileiro S.A. - Petrobras, Rio de Janeiro (BR)

(72) Inventor: Matheus Augusto ALVES CUGLIERI, Rio de Janeiro (BR)

(73) Assignee: Petróleo Brasileiro S.A. - Petrobras, Rio de Janeiro (BR)

(21) Appl. No.: 19/488,575

(22) PCT Filed: Jun. 7, 2024
(86) PCT No.: PCT/BR2024/050236

§ 371 Date: Nov. 26, 2025

(30) Foreign Application Priority Data
Jun. 9, 2023 (BR) 1020230113575

(51) Int. Cl. G06T 7/00; G06T 7/174; G06T 7/90

(52) U.S. Cl. CPC G06T 7/0004; G06T 7/174; G06T 7/90; G06T 2207/10056; G06T 2207/20081; G06T 2207/20084; G06T 2207/30181

(57) ABSTRACT

Described herein are methods for automating petrological interpretation using genetic interpretation logic. Banks of building elements and original mineralogical compositions for each building element are created. A photomicrograph is segmented by building elements, and each mineralogical composition identified for each pixel within the original mineralogical composition of the building element is collated. The mineralogical compositions of the pixels within the perimeter of each identified building element are compared. A porosity, mineralogical content, and available building element data are identified and used to create a distribution map. The data from the distribution map is collated with the banks of building elements and original mineralogical compositions for each building element. The results are arranged as a geological process quantification table or a geological process distribution map.

### CROSS-REFERENCE TO RELATED APPLICATIONS

This application is a U.S. national stage application under 35 U.S.C. § 371 of International Application No. PCT/BR2024/050236, filed Jun. 7, 2024, which claims priority of Brazilian Application No. 1020230113575, filed Jun. 9, 2023. The entire contents of each priority application are incorporated herein by reference.

### FIELD

The present invention pertains to the field of technologies responsible for classifying parameters related to the lithological characterization, particularly at the microscopic scale; more specifically, regarding the characterization by means of genetic interpretations.

### BACKGROUND

Geological information obtained through petrography is essential for understanding any lithological unit. Currently, this work is carried out manually; that is, through the analysis and annotation of observations in different databases by experts in the field. This work, given the level of detail required, is time-consuming, making its ideal execution (with qualitative and quantitative assessments of the identified phases and interpreted processes) unfeasible, depending on the number of slides available for study.

The microscale approach, conducted through petrographic studies, provides support for lithological classification and characterization of the different units based on petrological and petrophysical attributes.

The complexity of petrographic description work, which requires technical expertise and time to acquire and interpret data, can lead to delays in subsequent processes, which depend, to some extent, on the data generated through the microscopic analysis. This leads to delays and hinders data integration. In addition, failure to meet expectations due to the complexity of this petrographic work contributes to its increasingly limited use in activities that require agility, often associated with extractive industries (such as hydrocarbons). This reduced use of this type of data, in turn, makes subsequent steps more limited, as decisions are made without considering information at all scales of investigation.

This generates as a consequence partial or sometimes erroneous information, which is used and replicated in subsequent steps of the workflow, or a limitation to the potential data to be obtained, which goes against the goal of optimizing rock sample collection, a time-consuming and costly activity. This outlined context is a rough outline of the problems encountered in the current context, but it can be replicated for any activity involving massive sample collection and the need for detailed petrographic studies.

In addition to lithological classification, microscopic analysis allows for the acquisition of other interpretive data related to the processes that form the mineral phases.

These processes, expressed in the form of mineralogical variation, crystallinity, and/or porosity allocation, are usually based on knowledge acquired through the observation of multiple specimens of the lithological textures, bibliographic research, and, when possible, the application of analogous processes.

Obtaining reliable results requires extensive knowledge in the field, which demands years of dedication and specialization. In addition to specific expertise, the interpretations require time to be developed, which is sometimes limited. The large number of rock slides produced and the urgency of the petrographic results to support decisions in the Oil Exploration and Production chain make the service incomplete, heterogeneous, and often subject to revision and standardization, which demands even more man-hours and rework by experts.

### STATE OF THE ART

The search for the history of the invention in question led to some documents that disclose matters within its technological field. However, these documents still identified one or more of the aforementioned technical issues.

Document US20130325349 describes several embodiments for generating a refined classification of sedimentary facies corresponding to an oil or subsurface gas field or reservoir.

The method of US20130325349 may include analyzing a plurality of rock cores obtained from a plurality of wells drilled in the reservoir or field and analyzing a plurality of different well logs obtained from the plurality of wells, and determining an initial sedimentary facies classification for at least portions of the reservoir or oil or gas field. It is then determined whether at least one diagenetic, heavy, light, or anomalous mineral is detected in at least some of the analyzed rock cores and, if so, whether at least one type of well log among a plurality of different well types is capable of substantially and previously identifying the presence of at least one diagenetic, light, or anomalous mineral. Then, the initial sedimentary facies classification is reanalyzed and reclassified to produce a refined sedimentary facies classification.

In summary, US20130325349 addresses to a method used for automated lithofacies classification, which is not the purpose of the method of the present invention. It is worth highlighting that the automated rock classification involves generating a product that is essentially different from the product to be generated by the proposed method. In addition, the data used by the reference include well log curves, through the verification of similarities between the different curves to establish estimates regarding the lithofacies. This does not fall within the scope of the present invention, which aims at using photographs of petrographic slides to obtain the aforementioned data. In other words, the mentioned products materialize the action of geological processes that are relevant to the conception of the rock textures, but do not necessarily condition the lithological classification (although they may condition the properties of these lithologies, such as reservoir characteristics or characterization of compositional and densimetric properties, etc.).

Document CN105651962 provides a method for recognizing diagenetic facies. The diagenetic facies recognition method comprises obtaining diagenetic facies characterization parameters and obtaining the integrative module of diagenesis, according to the obtained diagenesis intensity, in which obtaining the diagenetic facies characterization parameters comprises measuring the diagenesis intensity or diagenesis intensity and diagenetic mineral content. Or, further, the diagenetic facies recognition method comprises obtaining diagenetic facies characterization parameters, and obtaining the diagenetic facies characterization parameters comprises measuring the diagenetic mineral content. According to the obtained integrative module of diagenesis or diagenetic mineral content, a type of reservoir diagenetic facies can be recognized. The time-consuming and costly technical issues of the rock core analysis process of the prior art can be resolved, and a quantitative assessment of the reservoir's diagenetic facies can be achieved.

CN105651962, by means of the analysis of the diagenetic facies, aims at defining the mineral composition, although the way this classification is made is not well established. This is because the aforementioned document does not comment on the presence of Building Elements (or similar structures), which is one of the cornerstones of the method proposed in the present invention. The discretization of the lithofacies solids into two domains (compositional and “building elements”) is what allows the recognition and nature of the generated products.

Furthermore, CN105651962 cites “diagenetic facies,” which appears to restrict the application of the method and makes its application and reproduction in other contexts very complicated. Finally, the method in the aforementioned document associates a mineralogical composition with a geological process, a relation that is quite imprecise (and perhaps applicable only to a specific context). The method proposed in the present invention, in turn, with the purpose of representing the existing geological complexity, brings more complex and representative relations to the geological context, which makes the generated method more precise and accurate.

One embodiment of the invention of CN112505154 provides a mineral component content analysis of a shale reservoir and a lithofacies identification characterization method, which comprises the following steps: establishing an analytical inversion model of the mineral component content in the shale reservoir and solving the analytical inversion model to obtain analytical calculation results for all minerals; and obtaining the lithofacies recognition result by means of a mineral component decision tree based on the calculation result of the analysis for each mineral. According to the embodiment of the invention, conventional logging data are used to establish the inversion analysis model of the mineral component of the shale reservoir; the program is compiled using the blind-source simulated annealing method to combine the iterative solution, and the relative volume content of each mineral is analyzed, and the comparison with the central analysis result is carried out.

CN112505154 claims a method for analyzing the mineral component content of a shale reservoir and identifying lithofacies, comprising the steps of: establishing an analytical inversion model for the mineral component content in the shale reservoir and solving the analytical inversion model to obtain analytical calculation results for all minerals; and obtaining the lithofacies recognition result by means of a decision tree for the mineral components based on the analysis calculation result for each mineral.

The aforementioned document's scope is the classification of “shale” facies in terms of constituents. It appears that the definition of “constituent” as used refers to the mineralogical composition used in the method proposed in the present invention. In addition to this restricted scope, which is not applicable (and not reproducible, given the specificities of this rock type) to most situations, the lack of identification of the constituents (referred to, in the present invention, as “Building Elements”) makes it impossible to obtain information about the genetic processes targeted by the method proposed in this invention.

Izadi et al. (2017) disclose an intelligent system for identifying minerals in thin sections, proposed based on RGB and HSI color spaces and texture features in flat and cross-polarized light. The proposed system has two phases for mineral identification. In phase #1, the segmentation phase, 12 color components are extracted for each pixel and, using an incremental clustering algorithm, several mineral clusters, including the index mineral, are produced. Subsequently, in phase #2, the identification phase, the resulting mineral clusters are identified using a cascade classification approach.

The first level of the cascade includes a set of artificial neural networks (ANNs) corresponding to the number of input minerals that are trained based on color components. In the first level, minerals that exhibit different colors in flat or cross-polarized light are identified. The second level of the cascade includes an ANN that is trained based on texture features in flat and cross-polarized light images.

In the second level, the minerals that are indistinguishable based on color components, both in the natural light optical system and with crossed polarizers (orthoscopic system), are identified (they are rejected in the first level of the cascade). The system's final output is the name and number of minerals, the limit and percentage of each mineral in the thin section, and, eventually, the name of the probable target rock. The system is capable of recognizing 23 igneous minerals of test with an overall accuracy of 93.81%. The system can be used in important applications that require a real-time identification and segmentation map, such as petrography and NASA Mars Exploration.

The method proposed in the aforementioned paper, applicable to specific rock types, is based on the identification of different mineralogical types (equivalent to the mineralogical identification, one of the two data domains collected in the method proposed by the present invention) through different levels of algorithms capable of, by means of the analysis of color patterns, estimating the classification of minerals and obtaining quantitative data. At no point does the method in the aforementioned paper highlight the distribution of these mineralogies in the structural elements that compose the rock, which makes it impossible to obtain information such as that intended by the method of the present invention.

In this way, the combination of CN112505154, or any of the cited references, with the aforementioned paper, for example, would not produce the intended effect of the method of the present invention, since it does not contain sufficient subsidies to address to the petrographic data according to the two domains-Mineralogical Composition and composition of structures (Building Elements).

In short, a technician skilled on the subject, in possession of any combination of the aforementioned documents, would not have the subsidies to obviously provide a method such as that of the present invention, since no method would include a step capable of collating the data collected with the structured database that was created from the Building Elements and Expected Mineralogical Composition, for each set of pixels identified as a given Building Element.

### SUMMARY

The proposed method aims at acquiring qualitative and quantitative data on the different petrographic constituents of the rock samples by means of automated recognition, quantification, and segmentation mechanisms, as well as at articulating this information to construct rapid interpretations that generate added value to the petrographic service and support decisions at various steps of the Oil Exploration and Production chain. The automated formulation of genetic processes seeks to provide interpretative parameters regarding the genesis and modification of the studied deposits, in order to support geological interpretations in the construction of static and dynamic models.

### BRIEF DESCRIPTION OF THE FIGURES

To obtain a complete and comprehensive view of the object of this invention, the figures to which references are made are presented, as follows.

FIG. 1 illustrates a scheme of the definition of a mineral phase in a petrographic context.

FIG. 2 illustrates a scheme of the criteria used for the temporal interpretation of the different identified mineral phases.

FIG. 3 shows a representative diagram (not to scale) of the acquisition of the three different data domains: porosity, mineralogy, and Building Elements.

FIG. 4 shows a representative diagram (not to scale) demonstrating the articulation between the information obtained, in order to generate different attributes for each pixel of the image.

### DETAILED DESCRIPTION

The method described in the present invention for automating petrological interpretation by means of genetic interpretation logic comprises the steps of:
I) Creating and/or imputing an information bank (database) of the following elements: Building Elements and Original Mineralogical Composition.

Namely, “Building Elements” are the structures to be identified, with the aid of microscopy, in the petrographic slides (or photomicrographs). They comprise the “building blocks” of the rock's solid phase. They are formed by a series of crystals, crystal aggregates, or constituent fragments, with distinct and identifiable internal arrangements, external geometries, and paragenetic relations, through which a rock can be classified. In turn, “Original Mineralogical Composition” is the initial mineralogy of each Building Element. Some elements present a single possible original mineralogical composition, while others may present more than one composition.
II) Collating each found mineralogical composition, obtained from the mineralogical segmentation performed (by whatever mineralogical identification methodology used), with the Original Mineralogical Composition of the Building Element (introduced in step I). The identification of the Building Element with its original mineralogical compositions, when compared with the observed mineralogical composition (obtained through any mineralogical segmentation system used), allows for the following cases: i) if a mineral Y is identified within the perimeter of a Building Element with original composition X, it is interpreted as a process of replacement of X by Y; or, if a porosity is identified within this same perimeter, it is interpreted as a dissolution process; ii) if a mineral X is identified within the perimeter of a Building Element with original composition X, it is interpreted as the Building Element still retains its original composition and structure.
III) Identifying and arranging the porosity, mineralogical content, and Building Elements data to create a distribution map, in which the same pixel position of said map stores information regarding the presence or absence of porosity, identifies the Mineralogical Composition, and establishes which Building Element it is inserted into.
IV) Collating the data obtained in step III) with the information database from step I), so that, for each set of pixels identified as a determined Building Element, the mineralogy/porosity information from said database is collated.
V) Arranging the results in outputs in a format chosen from: a geological process quantification table or a geological process distribution map.

It should be emphasized that, in step I), certain elements may have more than one Original Mineralogical Composition, which can lead to greater complexity in the relation between the data and require more advanced formulations for representing the interpreted processes. Table 1 below illustrates the correspondence between Building Elements and the Original Mineralogical Composition:

In step II), if a Building Element is originally composed of mineral X, if mineral Y is found within its perimeter, the associated process is called “Replacement [of X by Y] in [Building Element]”; if, instead of mineral X, porosity is identified, the associated process is “Dissolution of [Building Element]” in the set of instructions, as shown in Table 2 below:

This relation between mineralogies, as well as the process interpretation, are products of the data acquisition (manual or automated, depending on the user) and the interpretation of that lithological type, to be fed by the expert.

In step III, in the case of slide photomicrograph analyses, two approaches are suggested: the porosity and the mineralogical content can be obtained through pixel-by-pixel evaluation, using sets of instructions (algorithms) for porosity recognition and, within the solids content, which mineralogies exist, to be applied in the evaluation of the characteristics of each pixel (RGB color standard, for example).

Still in step III), for the recognition of Building Elements, a set of instructions (algorithm) for object detection is preferably used, trained to identify the perimeters of the Building Elements, as well as the pixels contained in these perimeters.

Each of the data domains must present a distribution map, referenced to each other, that is, the pixels of each distribution map must have correspondences in the pixels of the other two maps. In this way, the same pixel position acquires three more pieces of information (or labels): whether it is porosity or not; if it is not porosity, which is the identified mineralogy, and within which Building Element it is inserted. Just as important as the efficient recognition of these different constituents is the referencing of these three data domains, so that each pixel of the petrographic image acquires the following characteristics: “it is a pore/it is not a pore”; if it is not a pore: “is it the mineral “xx””; “it is contained within the perimeter of the Building Element “aaa””.

In step IV), depending on the situation, the relations established in step II) will indicate the different interpreted processes, as shown in Table 3 below:

For each Building Element identified, the original mineralogical content (“expected” content) is collated with the mineralogical content found by means of the proposed segmentation. If the mineralogical contents match, the found mineral phase (“Building element+mineralogy”) is the preserved original one, and if the mineralogical contents differ, a set of instructions implemented by the database provides a genetic interpretation relevant to the relation.

With respect to step V), it is worth emphasizing that the process quantification tables represent the relation between pixels in which certain processes are identified and the total number of pixels in the image, to estimate the intensity of the geological processes, and further the relation between pixel mineralogy and processes interpreted for each pixel, to estimate the genesis of the mineralogies, etc.). The process distribution maps spatially associate the interpreted process labels for each pixel.

For the genetic interpretation, knowledge of two types of data from the petrographic evaluation is necessary: the mineralogical/porosity distribution; and the arrangement of Building Elements.

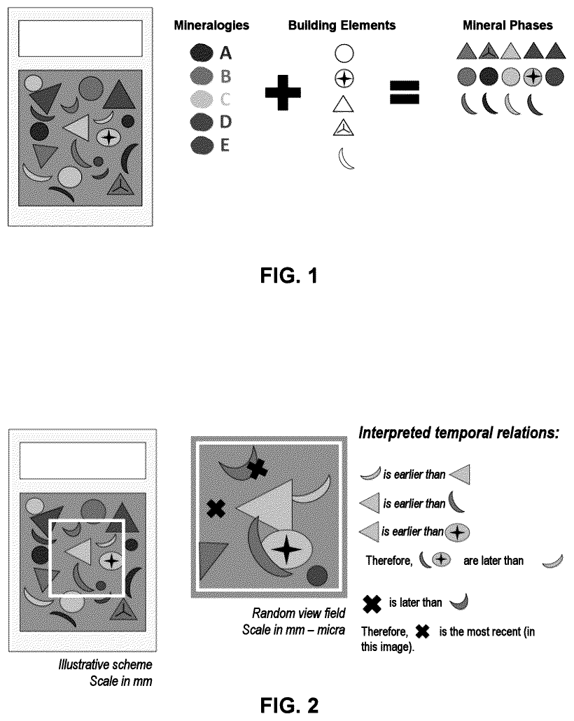

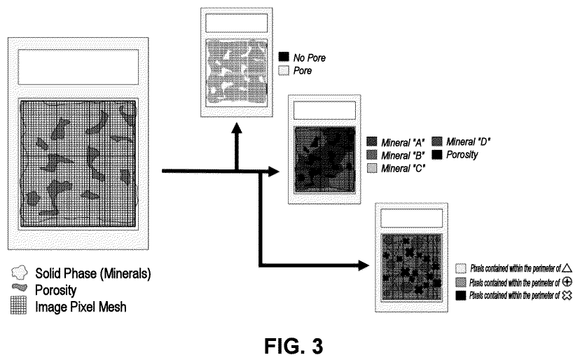

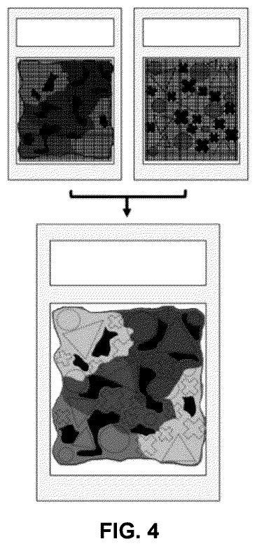

The connection between these two domains of data obtained substantiate the concept of “Mineral Phase,” which is, in fact, the building block of the lithological textures and records, together with the existing porosity, the genetic history of the sampled lithology, as represented in FIG. 1.

Genetic history refers to the succession of geological processes that occurred from the formation of the sediment until its microscopic investigation, with a focus on the diagenetic processes, the main agents of modification of the lithological textures and, consequently, their permoporous characteristics.

The suggested processes are the result of the obtained interpretation of the mineral phases identified in the lithology. The spatial relation between these mineral phases reflects, among other things, the timing of these processes, which is fundamentally the paragenetic interpretation of the rock and supports decisions such as genetic classification, petrophysical characterization, and the construction of static models (FIG. 2).

It is reiterated that interpreting a geological process in petrography requires data collection and prior knowledge of the following: identification of the Building Element, the original mineralogy/crystallinity, and the current mineralogy/crystallinity.

In short, the method promotes standardization and speed of the basic genetic interpretations, saving the expert time and favoring increased productivity, without significant loss of quality of the generated data. In addition, the appropriate formulation of the method enables data generation by users who lack in-depth knowledge of the subject, although they benefit from the results. This is because, once the structured database is well-designed (developed by an expert), the quality control of the generated data is facilitated, as it is linked to the paragenetic relations completed, known, and developed by an expert group. In addition, the method proposed in the invention is fully customizable, as it allows the user to adapt their interpretations by formulating different relations that illustrate and support the same.

Those skilled in the art will value the knowledge presented herein and will be able to reproduce the invention in the presented embodiments and in other variations encompassed by the scope of the appended claims.

1. A method for automating petrological interpretation by means of genetic interpretation logic, comprising:
I) creating and/or imputing banks of building elements and original mineralogical composition for each building element;
II) in a studied photomicrograph, segmenting by building elements (identify, delimit a perimeter, and highlight an area occupied by each building element); collating each mineralogical composition identified for each pixel with the original mineralogical composition of the building element; the mineralogical compositions of pixels within the perimeter of each identified building element will be compared (between the original mineralogical composition of the building element and a mineralogical composition identified through any mineralogical segmentation feature); if a mineral Y is identified within the perimeter of a building element with original composition X, it is interpreted as a process of replacement of X by Y, or, if a porosity is identified within this same perimeter, it is interpreted as a dissolution process; if a mineral X is identified within the perimeter of a building element with original composition X, it is interpreted as no significant alteration of the building element;
III) identifying and establishing a porosity, mineralogical content, and available building elements data to create a distribution map, in which the same pixel position on said map stores information regarding a presence or absence of porosity, identifies the mineralogical composition, and establishes which building element is inserted;
IV) collating the data obtained in step III) with an information database from step I), in which, for each set of pixels identified as a determined building element, the mineralogy/porosity information from the database is collated;
V) arranging results in outputs relevant to intended objectives, in a format chosen from: a geological process quantification table or a geological process distribution map.

2. The method for automating petrological interpretation of claim 1, wherein, in step I), each element can have more than one original mineralogical composition.

3. The method for automating petrological interpretation of claim 1, wherein, in step III), for analysis in slide photomicrographs, the porosity and mineralogical content can be obtained through pixel-by-pixel evaluation, using sets of instructions for recognizing porosity and, within the solids content, which mineralogies exist to be applied in the evaluation of the characteristics of each pixel.

4. The method for automating petrological interpretation of claim 1, wherein, in step III), for the recognition of building elements Building Elements, a set of instructions for object detection is preferably used, trained to identify the perimeters of the building elements, as well as the pixels contained in these perimeters.

5. The method for automating petrological interpretation of claim 1, wherein, in step III), the same pixel position acquires at least three pieces of information: whether it is porosity or not; if it is not porosity, which is the identified mineralogy; and within which building element it is inserted.

6. The method for automating petrological interpretation of claim 1, wherein, in step IV), for each building element identified, the original mineralogical content is collated with the mineralogical content found by the proposed segmentation.

7. The method for automating petrological interpretation of claim 6, wherein, in step IV), if the mineralogical contents match, the found mineral phase is the preserved original one, and, if the mineralogical contents differ, a set of instructions implemented by the database provides a genetic interpretation relevant to the relation.

8. The method for automating petrological interpretation of claim 1, wherein, in step V), the process quantification tables represent the relation between pixels in which determined processes were identified and the total number of pixels in the image, to estimate the intensity of the geological processes, and further the relation between the mineralogy of the pixels and the processes interpreted for each pixel.

9. The method for automating petrological interpretation of claim 1, wherein, in step V), the process distribution maps spatially associate the interpreted process labels for each pixel.
